(19) United States
(12) Patent Application Publication
(10) Pub. No.: US 2017/0065750 A1
(43) Pub. Date: Mar. 9, 2017

(54) IV ANTICOAGULANT TREATMENT SYSTEMS AND METHODS

(71) Applicant: Becton, Dickinson and Company, Franklin Lakes, NJ (US)

(72) Inventors: Yiping Ma, Layton, UT (US); Jeff Tai, Lehi, UT (US)

(21) Appl. No.: 15/251,513

(22) Filed: Aug. 30, 2016

Related U.S. Application Data
(60) Provisional application No. 62/213,920, filed on Sep. 3, 2015.

(51) Int. Cl. A61L 33/06; A61L 29/08; A61L 29/16; A61L 33/00

(52) U.S. Cl. CPC A61L 33/068; A61L 33/0029; A61L 29/085; A61L 29/16; A61L 33/0094; A61L 2300/42; A61L 2420/02

(57) ABSTRACT

An intravenous delivery system may have a plurality of components with interior surfaces that cooperate to define a fluid pathway through which fluid flows into a body of a patient. One or more anticoagulant coatings may reside on one or more of the interior surfaces to restrict blood clot formation in the fluid pathway. Manufacture of the intravenous delivery system may commence with provision of the components and preparation of an anticoagulant solution. The one or more interior surfaces may be exposed to the anticoagulant solution to form the anticoagulant coating. The anticoagulant coating may be caused to adhere to the one or more interior surfaces. The anticoagulant solution may be prepared by dissolving a triblock copolymer, such as PEO-PPO-PEO or PEO-PBD-PEO, in water. Irradiation may be applied to the anticoagulant coatings and interior surfaces to form covalent bonds.

### RELATED APPLICATIONS

This application claims the benefit of U.S. Provisional Application No. 62/213,920, filed Sep. 3, 2015, entitled IV ANTICOAGULANT TREATMENT SYSTEMS AND METHODS, which is incorporated herein by reference.

### BACKGROUND

The present invention is generally directed to systems and methods for intravenous (“IV”) delivery, by which fluids can be administered directly to a patient. More particularly, the present invention is directed systems and methods for manufacturing components of an intravenous delivery system. An intravenous delivery system according to the invention is used broadly herein to describe components used to deliver the fluid to the patient, for use in arterial, intravenous, intravascular, peritoneal, and/or non-vascular administration of fluid. Of course, one of skill in the art may use an intravenous delivery system to administer fluids to other locations within a patient's body.

One common method of administering fluids into a patient's blood flow is through an intravenous delivery system. In many common implementations, an intravenous delivery system may include a liquid source such as a liquid bag, a drip chamber used to determine the flow rate of fluid from the liquid bag, tubing for providing a connection between the liquid bag and the patient, and an intravenous access unit, such as a catheter that may be positioned intravenously in a patient. An intravenous delivery system may also include a Y-connector that allows for the piggybacking of intravenous delivery systems and for the administration of medicine from a syringe into the tubing of the intravenous delivery system.

Known catheter designs are subject to occlusion due to blood clot formation. Such occlusions may necessitate premature replacement of catheter components, requiring time and attention from health care professionals. Such occlusions are typically caused by the formation of a blood clot on the catheter surfaces, which eventually grows to a size sufficient to block fluid flow. In some instances, the clot may be flushed out of the catheter if flushing is carried out on a regular basis. In other cases, flushing may not remove the clots. Accordingly, conventional catheter flushing processes are not sufficiently reliable.

### BRIEF SUMMARY OF THE INVENTION

Embodiments of the present invention are generally directed to intravenous delivery systems that provide enhanced resistance to blood clot formation, and to methods for manufacturing such intravenous delivery systems. In one embodiment, the intravenous delivery system may have a plurality of components that have a plurality of interior surfaces that cooperate to define a fluid pathway through which medication flows into a body of a patient. The intravenous delivery system may also have one or more anticoagulant coatings on at least a first interior surface of the plurality of interior surfaces. The one or more anticoagulant coatings may restrict blood clot formation in the fluid pathway.

The one or more anticoagulant coatings may include a triblock copolymer. The triblock copolymer may be covalently bonded to the first interior surface. The triblock copolymer may be one of PEO-PPO-PEO and PEO-PBD-PEO. More specifically, the triblock copolymer may be designated by the trade name Pluronic® F108, from BASF Corporation. The one or more anticoagulant coatings may be attached to the first interior surface as one or more PEO brush layers, each having a thickness of less than 20 nanometers.

The first interior surface may be on a first component of the plurality of components. The first component may be a catheter tubing tip, catheter tubing, a catheter adapter, integrated extension tubing, or a Luer connect port.

The plurality of components may further have a plurality of exterior surfaces. The one or more anticoagulant coatings may be on substantially all of the plurality of interior surfaces, and on a first exterior surface of the plurality of exterior surfaces.

According to one method, an intravenous delivery system may be manufactured. The method may include providing a plurality of components of the intravenous delivery system such that the plurality of components have a plurality of interior surfaces that cooperate to define a fluid pathway through which fluid flows into a body of a patient. The method may further include preparing an anticoagulant solution and exposing at least a first interior surface of the plurality of interior surfaces to the anticoagulant solution to form one or more anticoagulant coatings that restrict blood clot formation in the fluid pathway. The method may further include causing the one or more anticoagulant coatings to adhere to at least the first interior surface.

Preparing the anticoagulant solution may include dissolving a triblock copolymer in water. The triblock copolymer may be PEO-PPO-PEO or PEO-PBD-PEO. Preparing the anticoagulant solution may further include dissolving Nisin and/or low molecular weight heparin in the water.

Causing the one or more anticoagulant coatings to adhere to at least the first interior surface may include forming a covalent bond between the one or more anticoagulant coatings and the first interior surface. Forming the covalent bond may include applying radiation to the one or more anticoagulant coatings and the first interior surface to induce formation of the covalent bond. Applying radiation to the one or more anticoagulant coatings and the first interior surface may include applying gamma irradiation, ultraviolet irradiation and/or electron beam irradiation to the one or more anticoagulant coatings and the first interior surface.

Exposing at least a first interior surface of the plurality of interior surfaces to the anticoagulant solution may include attaching, the one or more anticoagulant coatings to the first interior surface as one or more PEO brush layers. Each of the PEO brush layers may have a thickness of less than 20 nanometers. Further, exposing at least a first interior surface of the plurality of interior surfaces to the anticoagulant solution may include exposing, to the anticoagulant solution, a first component of the plurality of components, which may be a catheter tubing tip, catheter tubing, a catheter adapter, integrated extension tubing, or a Luer connect port.

Exposing at least a first interior surface of the plurality of interior surfaces to the anticoagulant solution may include exposing substantially all of the plurality of interior surfaces to the anticoagulant solution. The plurality of components may further include a plurality of exterior surfaces. The method may further include exposing a first exterior surface of the plurality of exterior surfaces to the anticoagulant solution.

According to one method, an intravenous delivery system may be manufactured. The method may include providing a plurality of components of the intravenous delivery system such that the plurality of components have a plurality of interior surfaces that cooperate to define a fluid pathway through which fluid flows into a body of a patient. The plurality of components may include at least catheter tubing, an adapter, and integrated tubing. The method may further include preparing an anticoagulant solution, and exposing at least a subset of interior surfaces of the plurality of interior surfaces to the anticoagulant solution to form anticoagulant coatings on the subset of interior surfaces that restrict blood clot formation in the fluid pathway. The subset of interior surfaces may be on at least the catheter tubing, the adapter, and the integrated tubing. The method may further include applying radiation to the anticoagulant coatings and the subset of interior surfaces to form covalent bonds between the anticoagulant coatings and the subset of interior surfaces.

Preparing the anticoagulant solution may include dissolving a triblock copolymer in water or other solutions such as saline. The triblock copolymer may be designated by the trade name Pluronic® F108, from BASF Corporation.

These and other features and advantages of the present invention may be incorporated into certain embodiments of the invention and will become more fully apparent from the following description and appended claims, or may be learned by the practice of the invention as set forth hereinafter. The present invention does not require that all the advantageous features and all the advantages described herein be incorporated into every embodiment of the invention.

### BRIEF DESCRIPTION OF THE SEVERAL VIEWS OF THE DRAWINGS

In order that the manner in which the above-recited and other features and advantages of the invention are obtained will be readily understood, a more particular description of the invention briefly described above will be rendered by reference to specific embodiments thereof that are illustrated in the appended drawings. These drawings depict only typical embodiments of the invention and are not therefore to be considered to limit the scope of the invention.

FIG. 1 is a plan view of an intravenous delivery system according to one embodiment;

FIG. 2 is a flowchart diagram illustrating a method of manufacturing the intravenous delivery system of FIG. 1, according to one embodiment;

FIG. 3 is a cross-sectional view of the intravenous delivery system, according to embodiments;

FIG. 4 is an enlarged cross-sectional view of a portion of the intravenous delivery system, according to some embodiments; and

FIG. 5 is an enlarged cross-sectional view of another portion of the intravenous delivery system, according to some embodiments.

### DETAILED DESCRIPTION OF THE INVENTION

The presently preferred embodiments of the present invention can be understood by reference to the drawings, wherein like reference numbers indicate identical or functionally similar elements. It will be readily understood that the components of the present invention, as generally described and illustrated in the figures herein, could be arranged and designed in a wide variety of different configurations. Thus, the following more detailed description, as represented in the figures, is not intended to limit the scope of the invention as claimed, but is merely representative of presently preferred embodiments of the invention.

Moreover, the Figures may show simplified or partial views, and the dimensions of elements in the Figures may be exaggerated or otherwise not in proportion for clarity. In addition, the singular forms “a,” “an,” and “the” include plural referents unless the context clearly dictates otherwise. Thus, for example, reference to a terminal includes reference to one or more terminals. In addition, where reference is made to a list of elements (e.g., elements a, b, c), such reference is intended to include any one of the listed elements by itself, any combination of less than all of the listed elements, and/or a combination of all of the listed elements.

The term “substantially” means that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

As used herein, the term “proximal”, “top”, “up” or “upwardly” refers to a location on the device that is closest to the clinician using the device and farthest from the patient in connection with whom the device is used when the device is used in its normal operation. Conversely, the term “distal”, “bottom”, “down” or “downwardly” refers to a location on the device that is farthest from the clinician using the device and closest to the patient in connection with whom the device is used when the device is used in its normal operation.

As used herein, the term “in” or “inwardly” refers to a location with respect to the device that, during normal use, is toward the inside of the device. Conversely, as used herein, the term “out” or “outwardly” refers to a location with respect to the device that, during normal use, is toward the outside of the device.

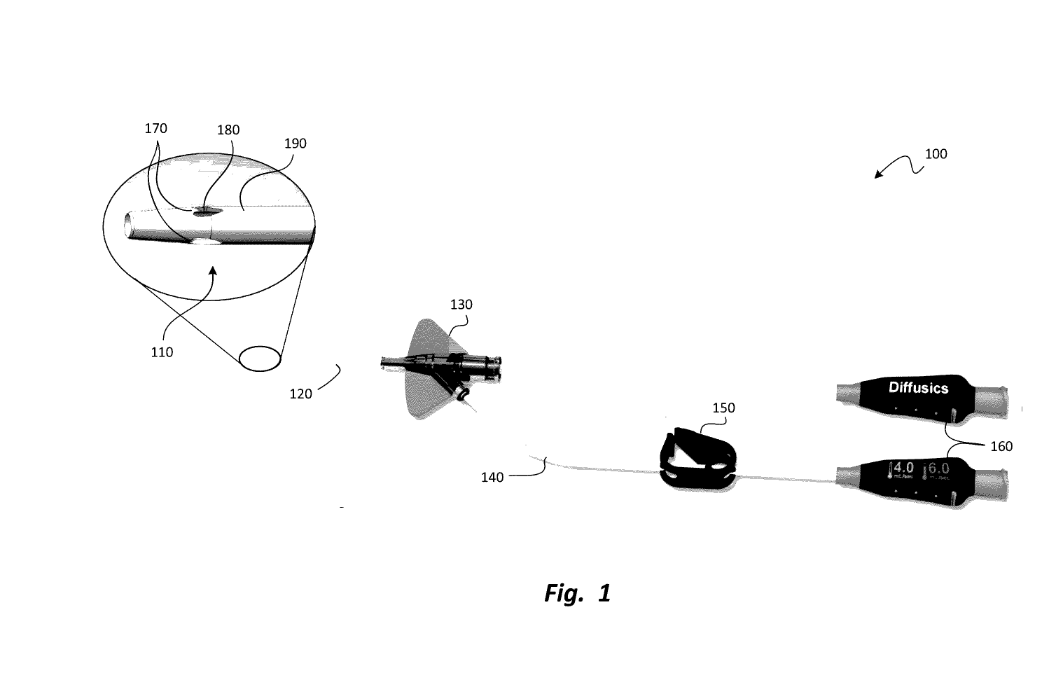

Referring to FIG. 1, a plan view illustrates an intravenous delivery system 100 according to one embodiment. The intravenous delivery system 100 may have a plurality of components that convey a fluid, such as medication or blood, to the body of a patient. The intravenous delivery system 100 may include various components, some of which are shown in FIG. 1, by way of example. As shown, the intravenous delivery system 100 may include a catheter tubing tip 110, catheter tubing 120, a catheter adapter 130, extension tubing 140, a clip 150, and a Luer connect port 160.

The Luer connect port 160 may be used to connect the intravenous delivery system 100 to a fluid source such as an IV bag or drip chamber (not shown). The clip 150 may be used to selectively reduce or stop fluid flow through the extension tubing 140 by compressing the extension tubing 140. The clip 150 may be selectively pressed into a clamping state, or released from the clamping state, by a user. The catheter adapter 130 may be used to facilitate introduction of another fluid into the intravenous delivery system 100, to be delivered to the patient along with the fluid flowing through the extension tubing 140. The catheter tubing 120 may be inserted through the patient's skin into the part of the body into which the fluid is to be administered, for example, into a blood vessel. The catheter tubing tip 110, which may be a tapered and sharpened tip of the catheter tubing 120, may be used for penetration of the tissue to access the fluid delivery site, and may reside in the fluid delivery site during delivery of the fluid to the patient.

As shown, the catheter tubing tip 110 may have a plurality of diffuser holes 170, which may enable the fluid to flow from the catheter tubing tip 110 in multiple directions, thereby diffusing fluid flow. The catheter tubing tip 110 may have an interior surface 180, which can be seen through the diffuser holes 170 and helps define a fluid pathway through the catheter tubing tip 110. Further, the catheter tubing tip 110 may have an exterior surface 190, which faces outward and contacts the tissue of the patient during introduction of the catheter tubing tip 110 into the fluid delivery site and remains in contact with the tissue during delivery of the fluid.

Like the catheter tubing tip 110, each of the other components of the intravenous delivery system 100, with the exception of the clip 150 (i.e., the catheter tubing 120, the catheter adapter 130, the extension tubing 140, and the Luer connect port 160) may have an inter surface and an exterior surface. The various interior surfaces of the fluid-conveying components of the intravenous delivery system 100 (the catheter tubing tip 110, the catheter tubing 120, the catheter adapter 130, the extension tubing 140, and the Luer connect port 160) may cooperate to define a fluid pathway through which fluid flows through the intravenous delivery system 100, into the body of the patient.

These interior surfaces may be in contact with blood and/or other fluids, such as the fluid to be administered to the patient, which may potentially cause blood clot formation. Further, some of the exterior surfaces, such as the exterior surface 190 of the catheter tubing tip 110 and the corresponding exterior surface of the catheter tubing 120, may be in contact with blood and/or other fluids within the body of the patient. Accordingly, these interior and exterior surfaces are locations at which blood clots may adhere and grow. Such blood clots may occlude blood flow.

Accordingly, it may be desirable to provide an anticoagulant coating on some or all of these interior and exterior surfaces. In some embodiments, all of the interior surfaces and exterior surfaces of all components of the intravenous delivery system 100 may have an anticoagulant coating. In other embodiments, only the interior and exterior surfaces of the components of the intravenous delivery system 100 that convey fluid (the catheter tubing tip 110, the catheter tubing 120, the catheter adapter 130, the extension tubing 140, and the Luer connect port 160) may have the anticoagulant coating.

In yet other embodiments, all of the interior surfaces and only some of the exterior surfaces of the fluid-conveying components of the intravenous delivery system 100 have the anticoagulant coating. Only the exterior surfaces expected to contact bodily or delivered fluid may be coated. For example, along with the interior surfaces, the exterior surface 190 of the catheter tubing tip 110 and the corresponding exterior surface of the catheter tubing 120 may have the anticoagulant coating.

In still other embodiments, all of the interior surfaces, and none of the exterior surfaces, of the fluid-conveying components of the intravenous delivery system 100 may have the anticoagulant coating. In yet other embodiments, only some of the interior surfaces of the fluid-conveying components of the intravenous delivery system 100 may have the anticoagulant coating. In still other embodiments, only one interior surface of one fluid-conveying component of the intravenous delivery system 100 may have the anticoagulant coating. For example, only the interior surface 180 of the catheter tubing tip 110 may have the anticoagulant coating. In embodiments in which not all interior surfaces have the anticoagulant coating, only the interior surface(s) deemed to be at greatest risk for blood clot formation and/or occlusion may be coated.

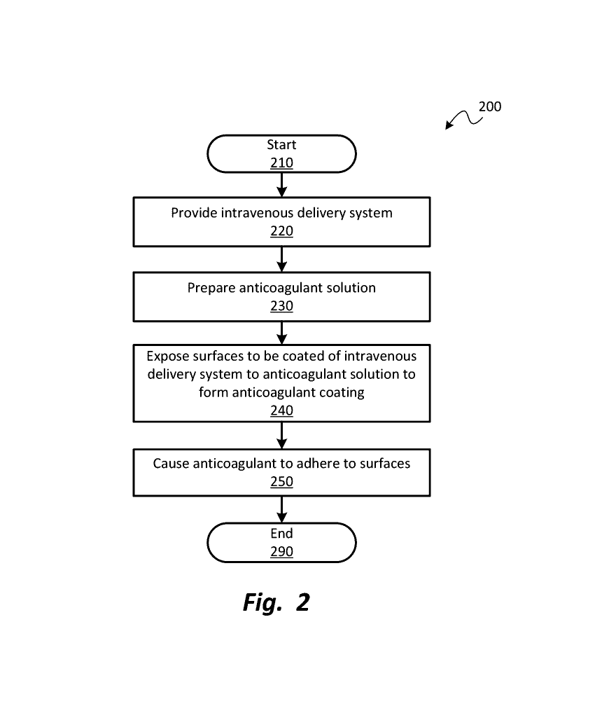

The intravenous delivery system 100 is merely exemplary. Those of skill in the art will recognize that, in other embodiments, various components of the intravenous delivery system 100 may be omitted, replaced, and/or supplemented with other intravenous delivery system components known in the art. The anticoagulant coating may be formed in a wide variety of ways. Some exemplary manufacturing methods will be shown and described in connection with FIG. 2.

FIG. 2 is a flowchart diagram illustrating a method 200 of manufacturing an intravenous delivery system according to one embodiment. The method 200 will be described in conjunction with the intravenous delivery system 100 of FIG. 1, as though used to manufacture the intravenous delivery system 100. However, those of skill in the art will recognize that the method 200 may be used to manufacture a wide variety of intravenous delivery system besides the intravenous delivery system 100 of FIG. 1, within the scope of the present disclosure. Similarly, the intravenous delivery system 100 of FIG. 1 may be made through the use of a variety of other methods, aside from the method 200 of FIG. 2, within the scope of the present disclosure.

The method 200 may start 210 with a step 220 in which the intravenous delivery system 100 is provided. The various components of the intravenous delivery system 100 (or other components, in the event that the method 200 is used to manufacture a different intravenous delivery system) may be manufactured through the use of any methods known in the art. The components of the method 200 may optionally be coupled together (for example, in the manner illustrated in FIG. 1) prior to undertaking further steps.

In a step 230, an anticoagulant solution may be provided. The anticoagulant solution may be formed, for example, by mixing an anticoagulant with water. A variety of anticoagulants may be used. In some embodiments, the anticoagulant may be a triblock copolymer. Some exemplary triblock copolymers include PEO-PPO-PEO and PEO-PBD-PEO, where PEO is polyethylene oxide, PPO is polypropylene oxide, and PBD is polybutadiene. More specifically, the triblock copolymer may be of a type sold under the name of Pluronic®, marketed by BASF Corporation. Yet more specifically, the triblock copolymer may include Pluronic® F 108, Pluronic® F 68, and/or Pluronic® F 127. In some embodiments, an end-activated group Pluronic® (E.G.A.P.) may be used.

These and other anticoagulants that may be used within the scope of the present disclosure may adhere to the surfaces to be coated via adsorption, and may “self-arrange” on the surfaces to be coated. For example, in the case of the triblock copolymers mentioned above, the PEO component of these molecules may be hydrophilic, while the central molecule (PPO or PBD) may be hydrophobic. The PPO or PBD domains, as hydrophobic molecules, may self-arrange on the surfaces to be coated in response to contact of the anticoagulant solution with the surfaces to be coated. The PEO domains, as hydrophilic molecules, may point away from the surfaces to be coated, thereby forming a “PEO brush layer.” The presence of the PEO brush layer may inhibit adsorption of (serum) proteins and/or aggregation of platelets on the interior surfaces that have been coated, thereby delaying and/or eliminating blood clot formation on those surfaces.

Various concentrations of the triblock copolymer may be dissolved in the water. In some embodiments, the concentration of the triblock copolymer may range from about 1 mg/mL of water to about 20 mg/mL of water. More specifically, the concentration of the triblock copolymer may range from about 2 mg/mL of water to about 10 mg/mL of water. Yet more specifically, the concentration of the triblock copolymer may range from about 3 mg/mL of water to about 7 mg/mL of water. Still more specifically, the concentration of the triblock copolymer may be about 5 mg/mL of water.

The actual concentration of anticoagulant in the anticoagulant solution may be dependent upon the manner in which the anticoagulant is to be applied to the surfaces to be coated in subsequent steps, the surface area of these surfaces, the particular type of anticoagulant used, and/or other factors. Thus, the concentration of anticoagulant in the anticoagulant solution may be tuned to the specific manufacturing process. The key may be to ensure that a sufficient quantity of anticoagulant is present in the anticoagulant solution to coat all surfaces to be coated with the desired coverage area. It may be acceptable to use a higher concentration of the anticoagulant because any suspended molecules that remain after adherence of the anticoagulant to the surfaces to be coated may be eluted away from the coated surfaces.

In some embodiments, the anticoagulant solution may be applied so as to provide a very thin PEO brush layer, for example, ranging in thickness from 1 nm to 20 nm in thickness. More specifically, the PEO brush layer may range in thickness from 5 nm to 15 nm in thickness. Yet more specifically, the PEO brush layer may range in thickness from 8 nm to 12 nm in thickness. Still more specifically, the PEO brush layer may be about 10 nm in thickness.

In some embodiments, the anticoagulant solution may also include an anticoagulant additive to enhance the anticoagulant properties. Many different anticoagulant additives may be used within the scope of the present disclosure. One example is low molecular weight heparin (LMWH). The anticoagulant additive may be dissolved in water or other solutions after the formation of the tri-block copolymer coating on the device surface. The anticoagulant additive will then be entrapped in the triblock copolymer brush layer. Various concentrations of LMWH may be used. As with the triblock copolymer, the concentration of the anticoagulant additive in the anticoagulant solution may be tuned to the specific manufacturing process, with the possibility of eluting away excess suspended molecules.

The anticoagulant and/or the anticoagulant additive, as applicable, may be dissolved in the water according to any known procedure to form the anticoagulant solution. The step 230 may then be complete.

Once the anticoagulant solution has been prepared, the method 200 may proceed to a step 240 in which the surfaces of the intravenous delivery system 100 to be coated are exposed to the anticoagulant solution. This may be done in a wide variety of ways.

According to one method, a “fill and drain” method may be used. The intravenous delivery system 100 may be filled with the anticoagulant solution, for example, through the use of a syringe containing the anticoagulant solution. The tip of the syringe may be inserted into one of the open ends of the intravenous delivery system 100 (for example, the end of the catheter tubing tip 110 or the end of the Luer connect port 160). The other end of the intravenous delivery system 100 may be left open so that the anticoagulant solution passes through the intravenous delivery system 100 and exits the intravenous delivery system 100 through the open end. Alternatively, the other end of the intravenous delivery system 100 may be plugged so that the intravenous delivery system 100 is more likely to fill with the anticoagulant solution, thereby providing more complete exposure of the interior surfaces of the intravenous delivery system 100 to the anticoagulant solution.

The intravenous delivery system 100 may remain plugged until the anticoagulant solution has remained in contact with the surfaces to be coated for a predetermined length of time. If desired, the syringe may be removed, and the end to which it was coupled may also be plugged for convenience so that the intravenous delivery system 100 can easily be left in place while the anticoagulant adheres to the surfaces to be coated. The length of time needed may depend on the particular components of the anticoagulant solution, the surface area of the surfaces to be coated, the concentration of the various solutes in the anticoagulant solution, the ambient temperature, the hydrophobicity of the surface, and/or other factors.

In some embodiments, the anticoagulant may adsorb to the surfaces to be coated substantially immediately, requiring no significant resting time. In other embodiments, the anticoagulant solution may be left in contact with the surfaces to be coated for a few minutes, a few hours, or even a few days in order to provide sufficient time for the anticoagulant molecules to auto-arrange on and adhere to the surfaces to be coated. In some exemplary embodiments, the anticoagulant solution may be left to incubate for about four hours at room temperature (23° C.) to allow the auto-arrangement and adherence to occur.

As mentioned previously, it may be desirable to coat one or more of the exterior surfaces of the intravenous delivery system 100 in addition to one or more of the interior surfaces. In order to accomplish this, alternative exposure methods may be used. According to one alternative embodiment, the intravenous delivery system 100 may be dipped in the anticoagulant solution. The intravenous delivery system 100 may be dipped in its entirety in the anticoagulant solution; alternatively, only components of the intravenous delivery system 100 for which the interior and exterior surfaces are to be coated may be dipped. The intravenous delivery system 100 (or portions thereof) may remain immersed in the anticoagulant solution for the optimal period of time for adherence and auto-arrangement of the anticoagulant, as described previously.

These exposure methods are merely exemplary. Those of skill in the art will recognize that any known method whereby a surface can be exposed to the solute of a solution may be used to expose the surfaces of the intravenous delivery system 100 to be coated to the anticoagulant, and/or the anticoagulant additive. One exemplary alternative method is to spray the anticoagulant solution onto the surfaces to be coated. The spray may be a fine mist so as to atomize the anticoagulant solution, thereby providing relatively rapid and even coverage of the surfaces.

Once exposure is complete, the intravenous delivery system 100 may be removed from the anticoagulant solution and allowed to dry. As indicated previously, any excess suspended molecules (for example, the anticoagulant, the antibacterial additive, and/or the anticoagulant additive) may be eluted away from the surfaces to be coated.

The surfaces to be coated may now each have an anticoagulant coating formed by adherence and self-arrangement of the anticoagulant on the surfaces. The adherence of the anticoagulant coatings to the surfaces may be sufficient to prevent and/or resist blood clot formation during usage of the catheter. However, in some embodiments, it may be desirable to more securely bond the anticoagulant coatings to the surfaces to help the anticoagulant coatings to remain in place and/or extend the useful life of the anticoagulant coatings.

Accordingly, once the anticoagulant coatings have been formed on the surfaces to be coated, the method 200 may optionally proceed to a step 250 in which the anticoagulant coatings are caused to adhere to the surfaces that have been coated. This may be done in a variety of ways. According to some exemplary embodiments, covalent bonds may be formed between the triblock copolymers and the surfaces on which they reside. This may be done, according to some embodiments, by applying irradiation to the anticoagulant coatings and the surfaces on which they reside.

Irradiation may be applied according to a wide variety of procedures. Exemplary procedures include, but are not limited to, gamma irradiation, ultraviolet irradiation, and electron beam irradiation. Irradiation may be conducted for a time sufficient to cause the covalent bonds to form between the triblock copolymers and the surfaces to which they are applied. Irradiation may be conducted for a few minutes, a few hours, or even a few days in order to provide sufficient time for the covalent bonds to form. According to one example, the surfaces and anticoagulant coatings may be irradiated by a 60 Co source over eight days to a total dose of 80 kGv.

In the alternative to application of irradiation, any other known method may be used to form the covalent bonds. Such alternatives may include the addition of binders and/or other agents in the anticoagulant solution to cause or facilitate formation of the covalent bonds. Further, in other alternatives, other methods, such as application of thermal energy, may be used to strengthen adherence of the anticoagulant layers to the surfaces through the use of one or more mechanisms besides covalent bonding.

Once the anticoagulant coatings have been caused to adhere to the surfaces with sufficient strength, the surfaces and anticoagulant coatings may be rinsed with water or other rinsing agents to remove any loosely-bound triblock copolymers. The method 200 may then end 290. The intravenous delivery system 100 may be ready for use. The anticoagulant coatings may prevent or delay blood clot formation within the fluid path defined by the interior surfaces of the intravenous delivery system 100 and/or on the exterior of the intravenous delivery system 100, depending on where the anticoagulant coating has been applied.

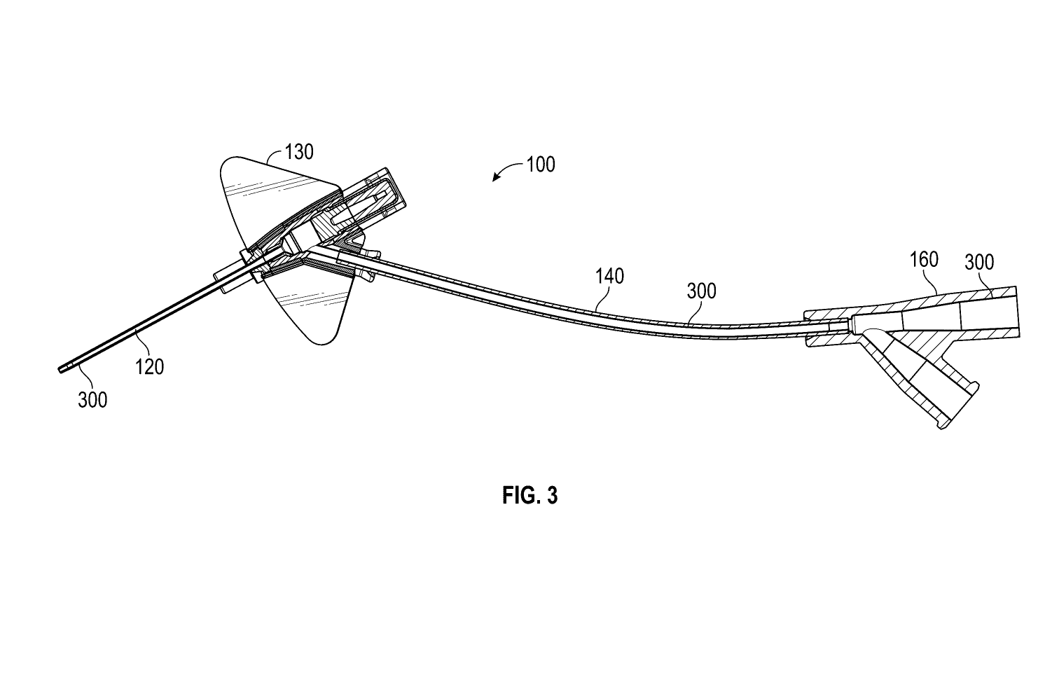

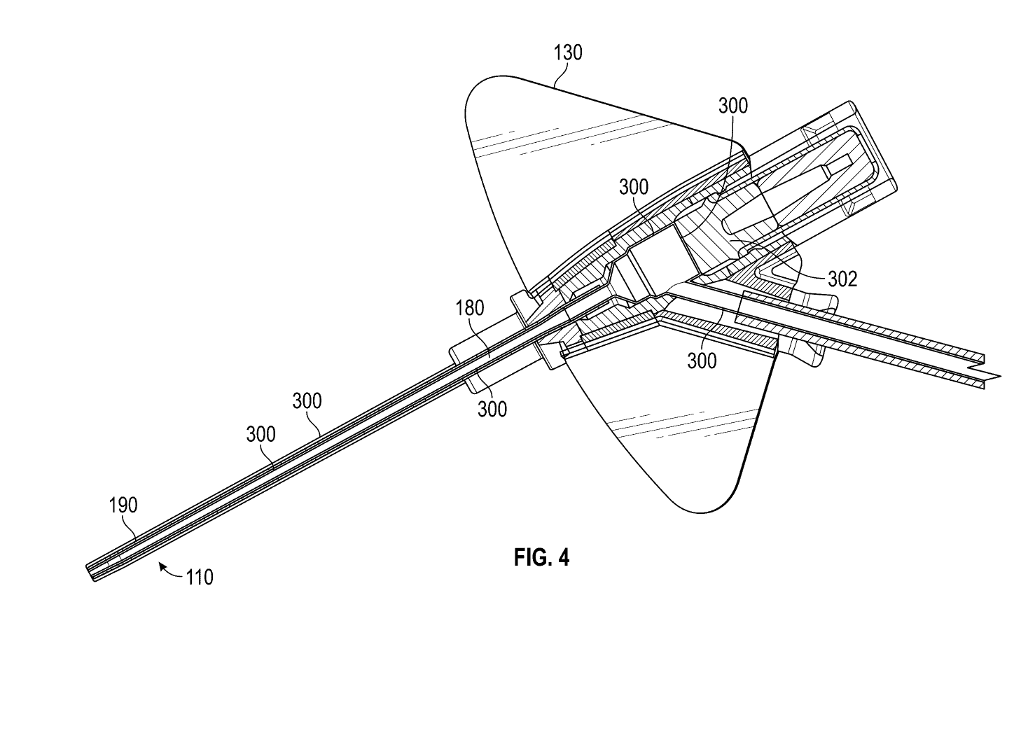

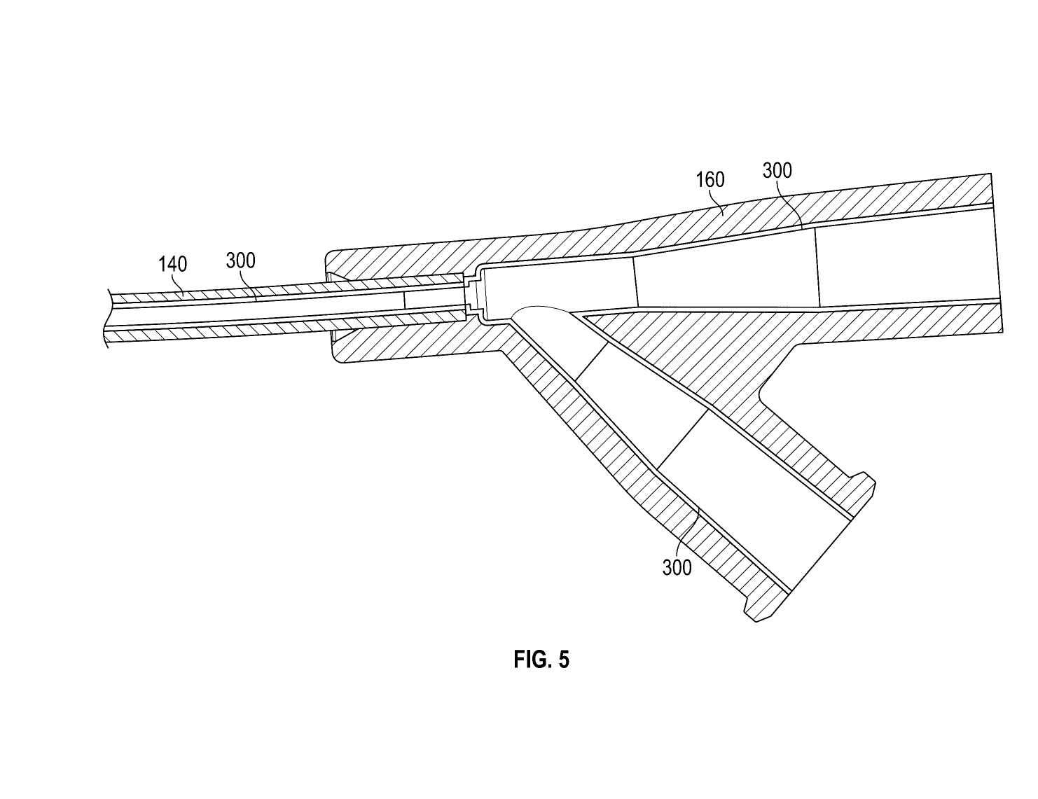

FIGS. 3-5 illustrate an anticoagulant coating 300 on a plurality of internal surfaces and at least one external surface of the intravenous delivery system 100. In some embodiments, the anticoagulant coating 300 may be applied to a distal surface of a septum 302.

We claim:

1. An intravenous delivery system comprising:
a plurality of components comprising a plurality of interior surfaces that cooperate to define a fluid pathway through which fluid flows into a body of a patient; and
one or more anticoagulant coating on at least a first interior surface of the plurality of interior surfaces, wherein the one or more anticoagulant coatings restrict blood clot formation in the fluid pathway.

2. The intravenous delivery system of claim 1, wherein the one or more anticoagulant coatings comprise a triblock copolymer.

3. The intravenous delivery system of claim 2, wherein the one or more anticoagulant coatings are covalently bonded to the first interior surface.

4. The intravenous delivery system of claim 2, wherein the triblock copolymer is selected from the group consisting of PEO-PPO-PEO and PEO-PBD-PEO.

5. The intravenous delivery system of claim 4, wherein the triblock copolymer is designated by the trade name Pluronic® F108, from BASF Corporation.

6. The intravenous delivery system of claim 4, wherein the one or more anticoagulant coatings are attached to the first interior surface as one or more PEO brush layers, each having a thickness of less than 20 nanometers.

7. The intravenous delivery system of claim 2, wherein the one or more anticoagulant coatings further comprise low molecular weight heparin.

8. The intravenous delivery system of claim 1, wherein the first interior surface is on a first component of the plurality of components, wherein the first component is selected from the group consisting of:
a catheter tubing tip;
catheter tubing;
a catheter adapter;
integrated extension tubing; and
a Luer connect port.

9. The intravenous delivery system of claim 1, wherein the plurality of components further comprise a plurality of exterior surfaces, wherein the one or more anticoagulant coatings are on substantially all of the plurality of interior surfaces, and on a first exterior surface of the plurality of exterior surfaces.

10. A method for manufacturing an intravenous delivery system, the method comprising:
providing a plurality of components of the intravenous delivery system such that the plurality of components comprise a plurality of interior surfaces that cooperate to define a fluid pathway through which fluid flows into a body of a patient;
preparing an anticoagulant solution;
exposing at least a first interior surface of the plurality of interior surfaces to the anticoagulant solution to form a anticoagulant coatings that restrict blood clot formation in the fluid pathway; and
causing the anticoagulant coatings to adhere to at least the first interior surface.

11. The method of claim 10, wherein preparing the anticoagulant solution comprises dissolving a triblock copolymer in water or other solution, wherein the triblock copolymer is selected from the group consisting of PEO-PPO-PEO and PEO-PBD-PEO.

12. The method of claim 11, wherein preparing the anticoagulant solution further comprises dissolving low molecular weight heparin or other anticoagulant drug molecule in the water or other suitable solution.

13. The method of claim 11, wherein causing the anticoagulant coatings to adhere to at least the first interior surface comprises forming a covalent bond between the anticoagulant coatings and the first interior surface.

14. The method of claim 13, wherein forming a covalent bond between the anticoagulant coating and the first interior surface comprises applying irradiation to the anticoagulant coating and the first interior surface to induce formation of the covalent bond.

15. The method of claim 14, wherein applying radiation to the anticoagulant coating and the first interior surface comprises applying a selection from the group consisting of gamma irradiation, ultraviolet irradiation and electron beam irradiation to the anticoagulant coating and the first interior surface.

16. The method of claim 10, wherein exposing at least a first interior surface of the plurality of interior surfaces to the anticoagulant solution comprises attaching the anticoagulant coating to the first interior surface as one or more PEO brush layers, each having a thickness of less than 20 nanometers.

17. The method of claim 10, wherein exposing at least a first interior surface of the plurality of interior surfaces to the anticoagulant solution comprises exposing, to the anticoagulant solution, a first component of the plurality of components selected from the group consisting of:
a catheter tubing tip;
catheter tubing;
a catheter adapter;
integrated extension tubing; and
a Luer connect port.

18. The method of claim 10, wherein exposing at least a first interior surface of the plurality of interior surfaces to the anticoagulant solution comprises exposing substantially all of the plurality of interior surfaces to the anticoagulant solution; and
wherein the plurality of components further comprise a plurality of exterior surfaces, the method further comprising exposing a first exterior surface of the plurality of exterior surfaces to the anticoagulant solution.

19. A method for manufacturing an intravenous delivery system, the method comprising:
providing a plurality of components of the intravenous delivery system such that the plurality of components comprise a plurality of interior surfaces that cooperate to define a fluid pathway through which fluid flows into a body of a patient, wherein the plurality of components comprises at least catheter tubing, an adapter, and integrated tubing;
preparing an anticoagulant solution;
exposing at least a subset of interior surfaces of the plurality of interior surfaces to the anticoagulant solution to form anticoagulant coating on the subset of interior surfaces that restrict blood clot formation in the fluid pathway, wherein the subset of interior surfaces is on at least the catheter tubing, the adapter, and the integrated tubing; and
applying irradiation to the anticoagulant coating and the subset of interior surfaces to form covalent bonds between the anticoagulant coating and the subset of interior surfaces.

20. The method of claim 19, wherein preparing the anticoagulant solution comprises dissolving a triblock copolymer in water, wherein the triblock copolymer is designated by the trade name Pluronic® F108, from BASF Corporation.
